(19) United States
(12) Patent Application Publication
(10) Pub. No.: US 2010/0303903 A1
(43) Pub. Date: Dec. 2, 2010

(54) METHODS OF ENHANCING SELECTIVE SEROTONIN REUPTAKE INHIBITOR EFFECTS IN MAMMALS

(75) Inventor: David HACKETT, Bucks (GB)

Correspondence Address: LUCAS & MERCANTI, LLP, 475 PARK AVENUE SOUTH, 15TH FLOOR, NEW YORK, NY, 10016, US

(73) Assignee: SHIRE LLC, Florence, KY (US)

(21) Appl. No.: 12/787,671

(22) Filed: May 26, 2010

Related U.S. Application Data
(60) Provisional application No. 61/181,177, filed on May 26, 2009.

(51) Int. Cl. A61K 31/4525; A61K 31/343; A61K 31/137; A61K 9/28; A61K 9/48; A61P 25/24

(52) U.S. Cl. 424/456; 514/469; 514/321; 514/647; 424/474

(57) ABSTRACT

The invention relates to methods of enhancing selective serotonin reuptake inhibitor effects in mammals. In particular, the invention provides methods for treating selective serotonin reuptake inhibitor dependent conditions such as depression. More specifically, the present invention relates to a method of increasing the antidepressant activity of a selective serotonin reuptake inhibitor (“SSRI”) by administering L-lysine-d-amphetamine in combination with an SSRI and to formulates containing the same. In a preferred aspect, the combination is administered in connection with a method of treating depression. One preferred SSRI is escitalopram. The preferred amphetamine prodrug is L-lysine-d-amphetamine.

### CROSS-REFERENCE TO RELATED APPLICATION

This application claims the benefit of priority from U.S. Provisional Patent Application Ser. No. 61/181,177 filed May 26, 2009, the contents of which are incorporated herein by reference.

### FIELD OF INVENTION

The invention relates to methods of enhancing selective serotonin reuptake inhibitor effects in mammals. In particular, the invention provides methods for treating selective serotonin reuptake inhibitor dependent conditions such as depression.

### BACKGROUND OF THE INVENTION

Depression is a serious illness that affects a person's family, work or school life, sleeping and eating habits, and general health. Its impact on functioning and well-being has been equated to that of major chronic medical conditions such as diabetes.

A person suffering from depression usually exhibits a very low mood that pervades all aspects of life. Depressed people may be preoccupied with thoughts and feelings of worthlessness, inappropriate guilt or regret, helplessness, hopelessness, and self hatred. Other symptoms include somnolence/fatigue, apathy, anhedonia, poor concentration and memory, withdrawal from social situations and activities, and thoughts of death or suicide. Insomnia is common: in the typical pattern, a person wakes very early and is unable to get back to sleep. Older depressed persons may have cognitive symptoms of recent onset, such as forgetfulness, and a more noticeable slowing of movements. In certain severe cases, depressed people may have symptoms of psychosis such as delusions or, less commonly, hallucinations, usually of an unpleasant nature.

After more than 50 years, the monoamine hypothesis of depression remains the most consistent hypothesis underlying antidepressant action. According to this hypothesis, depression arises from hypofunctioning of brain monoamine systems including the serotoninergic, noradrenergic, and/or dopaminergic pathways.

All currently available antidepressants are known to acutely enhance some aspect of monoaminergic function. Most are reuptake inhibitors of one or more of these monoamines.

Selective serotonin reuptake inhibitors (“SSRIs”) are a class of compounds typically used as antidepressants. SSRIs increase the extracellular level of serotonin (“5-HT”) by inhibiting its reuptake into the presynaptic cell, increasing the level of 5-HT available to bind to the postsynaptic receptor. They have varying degrees of selectivity for the other monoamine transporters, with pure SSRIs having only weak affinity for the norepinephrine (“NE”) and dopamine (“DA”) transporter.

Amphetamine belongs to a different class of drugs and it acts on the Central Nervous System (“CNS”) through two different pharmacological mechanisms. One mechanism consists in the inhibition of neuronal reuptake of NE and DA to prolong their concentration and time in the synaptic cleft. The second mechanism includes the ability to cause neuronal release of the three principle monoamine neurotransmitters DA, NE and 5-HT.

Many of the common residual symptoms of depression which lead to dysfunction, such as somnolence/fatigue, apathy, cognitive dysfunction and anhedonia, are the very symptoms that may be more susceptible to treatments with drugs like amphetamine which act through both mechanisms rather than with drugs exhibiting only the monoamine reuptake inhibition.

Recently, there have been calls for a new neurobiologically informed treatment strategy for targeting residual symptoms by augmenting antidepressants with agent capable of boosting specific neurotransmitters in the hypofunctioning brain monoamine pathways. An effective treatment for these negative symptoms remains however a substantial unmet need.

Thus, there is the need for a new treatment strategy for augmenting the antidepressant activity with agents capable of boosting specific neurotransmitters in the CNS. The present invention addresses this need.

### SUMMARY OF THE INVENTION

The combination of the invention seeks an improved treatment for depression and related cognitive disorders by combining an amphetamine prodrug such as L-lysine-d-amphetamine with one of a carefully selected group of SSRIs. The invention particularly seeks an improved treatment for depression. Not all SSRIs are effective in the sense of showing an augmentation effect with an amphetamine prodrug such as L-lysine-d-amphetamine; however we have found certain SSRIs to demonstrate an enhanced effect in combination with an amphetamine prodrug such as L-lysine-d-amphetamine. The prodrug is a conjugate in which amphetamine is covalently bound to an organic chemical species preferably such as an amino acid or a peptide containing from 1 to 10 amino acids. The amino acids are preferably independently selected at each occurrence from the naturally occurring amino acids.

The present invention relates to a method of increasing the monoamine levels in a mammal by administering an SSRI in combination with an amphetamine prodrug.

In another aspect, the present invention relates to a method of increasing the anti-depressant activity of a selective serotonin reuptake inhibitor (“SSRI”) by administering an amphetamine prodrug such as L-lysine-d-amphetamine in combination with an SSRI. The invention thus relates to a method of treating depression (and other disease states referred to in the literature which are known to be treatable with SSRIs alone) by administration to a mammal of the above combination. Other indications for which the combination may have efficacy include: autism, dementia, panic disorder, obsessive compulsive disorder (OCD), anxiety disorder and cognitive behavioural therapy.

In one embodiment of the present invention, the increased antidepressant activity and/or increased monoamine levels is provided by a combination of L-lysine-d-amphetamine and an SSRI selected from the group comprising: citalopram, escitalopram, paroxetine and sertraline.

In a preferred embodiment, the present invention relates to a method for increasing the antidepressant activity of escitalopram by administering L-lysine-d-amphetamine in combination with escitalopram.

In another preferred embodiment, the amount L-lysine-d-amphetamine administered in combination with the SSRI is an amount that is capable of causing only relatively minimal overt CNS effects.

The invention also relates to a formulation comprising an SSRI selected from the group comprising: citalopram, escitalopram, paroxetine and sertraline in combination with an amphetamine prodrug such as L-lysine-d-amphetamine and one or more pharmaceutically acceptable excipients.

The invention also relates to a combination of an amphetamine prodrug such as L-lysine-d-amphetamine and an SSRI selected from the group comprising: citalopram, escitalopram, paroxetine and sertraline for the treatment of depression in a mammal.

The invention also relates to a kit of parts comprising a combination of an amphetamine prodrug such as L-lysine-d-amphetamine and an SSRI selected from the group comprising: citalopram, escitalopram, paroxetine and sertraline in an oral dosage form. Frequently, the kit further includes dosing instructions for administration. The components are suitable for simultaneous, sequential or separate administration. The kit may include packaging to indicate the dosing regimen required.

### BRIEF DESCRIPTION OF THE DRAWINGS

FIG. 1 shows the effect of the combination of L-lysine-d-amphetamine with escitalopram on immobility time.

FIG. 2 shows the effect of the combination of L-lysine-d-amphetamine with escitalopram on latency to immobility.

FIG. 3 shows the effect of the combination of L-lysine-d-amphetamine with escitalopram in the rat light-box anxiety test.

### DETAILED DESCRIPTION OF THE INVENTION

As used herein the “anti-depressant effect” refers to the clinical assessment of improved symptoms or signs of depression.

As used herein, a “pharmaceutical composition” refers to any combination of two, three or more components, including the two active components which may be present in the same or different formulations. It may be in form of, for example, tablets, capsules, caplets, oral solutions and oral suspensions.

As used herein, a “mammal” preferably refers to humans although any mammal which could benefit from the combination therapy described herein is contemplated.

For all of the methods described herein, the identified compounds are contemplated to be employed in combination, simultaneously, or sequentially (e.g. in the same composition or in separate compositions).

It is thus to be understood that the term “combination” envisages the simultaneous, sequential or separate administration of the active components of the combination. Preferably the components are administered simultaneously. Conveniently, this normally occurs in one or more unit dosage forms containing both active components. Where the administration of those agents is sequential or separate, the delay in administering the second component should not be such as to lose the benefit of the synergistic or augmentation effect of the therapy.

In accordance with a preferred aspect of the invention, there is provided a method of increasing monoamine levels in mammals. In broad aspects, the method includes administering an effective amount a selective serotonin reuptake inhibitor, or a pharmaceutically acceptable salt thereof in combination with an amphetamine prodrug, or pharmaceutically acceptable salt thereof to a mammal in need thereof. The mammal is preferably a human patient.

The SSRIs included in some aspects of the invention are those well known to those of ordinary skill in the art and their therapeutic indications when administered alone are well documented. The approved indications for the stated SSRIs when administered in a lower than usual dosage in combination with an amphetamine prodrug in accordance with the invention also specifically forms part of the disclosure of the present invention. For brevity, the approved uses of the stated SSRIs is not listed here. Suitable SSRIs that may be used in the present invention include for example, citalopram, escitalopram, dapoxetine, femoxetine, fluoxetine, fluvoxamine, ifoxetine, paroxetine, sertraline, zimelidine, etc, and mixtures thereof. Preferred SSRIs are selected from the group comprising: citalopram, escitalopram, dapoxetine, femoxetine, fluvoxamine, ifoxetine, paroxetine, sertraline and zimelidine. More preferred SSRIs are selected from the group comprising: citalopram, escitalopram, paroxetine and sertraline. Even more preferred SSRIs are escitalopram or sertraline. In one embodiment, escitalopram is the preferred SSRI. In an alternate embodiment, sertraline is the preferred SSRI. The SSRIs are administered in amounts which are generally regarded as safe and effective for the treatment of depression or whatever clinical conditions the SSRI has been approved. The artisan is well aware of the dosing guidelines and the prescribing information available as part of the respective package inserts which provides the same is incorporated herein by reference.

In one preferred aspect of the invention, the method is carried out using escitalopram, i.e., the pure (S) enantiomer of racemic citalopram ((S)-1-[3-(dimethylamino)propyl]-1-(4-fluorophenyl)-1,3-dihydroisobenzofuran-5-carbonitrile), or salts thereof. The amount administered in this embodiment is an effective amount of escitalopram, i.e. an amount which is non-toxic but sufficient to demonstrate a cognizable anti-depressant effect in mammals, with the range being generally from about 1 mg to about 50 mg a day, preferably from about 5 mg to about 30 mg a day, and more preferably from about 10 mg to about 20 mg a day. In one preferred embodiment, the effective amount of escitalopram is based on the amount of escitalopram oxalate (Lexapro®) ranging from about 10 mg to about 20 mg/day. In an alternative embodiment, an escitalopram salt is administered in an amount substantially equivalent to the amount of escitalopram of escitalopram oxalate.

In an alternative aspect of the invention, escitalopram is administered to patients in an amount of from about 10% to about 95% relative to the amount of from about 10 mg to about 20 mg/day of escitalopram oxalate. In another alternative aspect of the invention, escitalopram is administered to patients in an amount of from about 20% to about 90%, from about 20% to about 80%, from about 40% to about 80%, from about 60% to about 80%, from about 20% to about 60%, or from about 20% to about 40% based on the dosage range of from about 10 mg to about 20 mg/day of escitalopram oxalate. The methods described herein are useful in reducing the dose level and/or frequency of dosage of SSRIs administered to patients in the treatment of depression. The methods described herein provide a means for treating depression with escitalopram with the dose level and frequency less than the currently effective daily doses. In one embodiment, escitalopram (e.g., escitalopram oxalate) can be administered in amounts less than the currently effective daily doses of from about 10 mg to about 20 mg/day. In another embodiment, escitalopram (e.g., escitalopram oxalate) can be administered in an amount of from about 10% to about 50% (e.g., from about 10% to about 45%, from about 20% about 45%, from about 30% to about 45%) of the currently effective daily dose of about 20 mg/day of escitalopram oxalate.

In a further aspect of the invention, the methods described herein provide a means for reducing adverse effects associated with SSRI therapy.

In another aspect of the invention, the methods described herein are carried out using sertraline or salt thereof. The amount administered in this embodiment is an effective amount of sertraline in a range of from about 25 mg to about 200 mg/day, from about 25 mg to about 150 mg/day, or from about 25 mg to about 75 mg/day (i.e., 25 mg, 50 mg, 75 mg, 100 mg, 150 mg, 200 mg/day). In one embodiment, sertraline hydrochloride salt (Zoloft®) is administered to patients in amounts ranging from about 25 mg to about 200 mg/day. In one preferred embodiment, the effective amount of sertraline salt is equivalent to 25 mg and 50 mg of sertraline.

In an alternative aspect of the invention, sertraline is administered to patients in an amount of from about 10% to about 95% relative to the amount of from about 25 mg to about 200 mg/day of sertraline. In another alternative aspect of the invention, sertraline is administered to patients in an amount of from about 20% to about 90%, from about 20% to about 80%, from about 40% to about 80%, from about 60% to about 80%, from about 20% to about 60%, or from about 20% to about 40% based on the dosage range of from about 25 mg to about 200 mg/day of sertraline. The methods described herein are useful in reducing the dose level and/or frequency of dosage of SSRIs administered to patients in the treatment of depressive disorder. The methods described herein provide a means for treating depressive disorder with sertraline with the dose level and frequency less than the currently effective daily doses. In one embodiment, sertraline can be administered in amounts less than the currently effective daily doses of from about 25 mg to about 200 mg/day. In another embodiment, sertraline can be administered in an amount of from about 5% to about 50%, from about 5% to about 10%, from about 8% to about 10%, from about 25% to about 32%, from about 25% about 32% of the currently effective daily dose of about 50 mg/day of sertraline.

The amphetamine prodrugs included in the methods of this invention preferably include amphetamine covalently bound to a chemical moiety, such as those as described in U.S. Pat. Nos. 7,223,735 and 7,105,486, (the '735 and '486 patents), the contents of which are incorporated herein by reference and these amphetamine conjugates specifically form part of the disclosure of the present invention in terms of the amphetamine component of the combination. The '735 and '486 patents describe covalent attachment of amphetamine and derivatives or analogs thereof to a variety of chemical moieties. The chemical moieties may include any substance which results in a prodrug form, i.e., a molecule which is converted into its active form in the body by normal metabolic processes. The chemical moieties may be for instance, amino acids, peptides, glycopeptides, carbohydrates, nucleosides, or vitamins and the unattached portion of the carrier/conjugate may be in a free and unprotected state, or in the form of an ester or salt thereof.

In one embodiment, the amphetamine is attached to a single amino acid which is either naturally occurring or a synthetic amino acid. In a preferred embodiment, the or each amino acid is a naturally occurring amino acid. The conjugate may contain from 1 to 10 amino acids in one preferred embodiment. In some preferred aspects of the invention, the amphetamine is attached to a dipeptide or tripeptide, which could be any combination of the naturally occurring amino acids and synthetic amino acids. In another preferred embodiment of the invention, the amino acids are selected from L-amino acids for digestion by proteases. One particularly preferred L-amino acid prodrug of amphetamine useful in the methods described herein is the L-lysine-d-amphetamine or (N-[(1S)-1-methyl-2-phenylethyl]-L-lysinamide, sold under the trademark Vyvanse® by Shire.

Surprisingly, we have found a beneficial effect for the combinations of the present invention. This allows, for example, for either or both components to be administered at a reduced level relative to the normal dosage when administered alone. The combination thus allows a reduced amount of SSRI to be given to a mammal such as a human patient. Thus, the adverse side-effects of SSRI administration may be ameliorated or removed using the combination therapy of the present invention. In the case of escitalopram, or indeed the other SSRIs, the ability to add L-lysine-d-amphetamine also allows one to reduce the toxicity of the SSRI. The SSRI can be administered in a lower amount than normal when in combination with an amphetamine and can be used to treat diseases normally treatable with a higher dose of the SSRI. Thus diseases treatable by, for example, escitalopram are treatable with the combinations of the present invention and form part of the invention.

The amount of amphetamine prodrug included is described as an effective amount, i.e. an amount which enhances the effectiveness of the SSRI agent in increasing monoamine levels while minimizing overt central nervous system effects which may be associated with the administration of some amphetamines to mammals. Stated another way, it is an amount which is capable of inducing antidepressant-like but not anxiogenic effect. The amount of amphetamine prodrug will vary somewhat, depending upon clinical conditions, but will be apparent to a clinician of ordinary skill without undue experimentation.

The dosing range of the L-lysine-d-amphetamine is normally in the range of 0.1 mg/kg to 75 mg/kg body weight per day in a single or divided doses. Similarly, the dosing range of the SSRI in the combination is in the range of 0.1 mg/kg to 75 mg/kg body weight per day.

Conveniently, the dosages are provided in unit dosage form containing both active components in the same form. The ratio of L-lysine-d-amphetamine to the SSRI (whether given in the same dosage form or separately) is in the range of 10:1 to 1:10 (weight: weight). More preferably, the ratio is in the range 5:1 to 1:2 and most preferably it is in the range 2:1 to 1:1. For purposes of illustration and not limitation, the amount of an amino acid prodrug of amphetamine which can be administered in accordance with the invention broadly ranges from about 5 mg to about 500 mg a day, and preferably from about 10 mg to about 250 mg a day. More preferably, the amount of L-lysine-d-amphetamine administered according to the present invention ranges from about 20 mg to about 70 mg a day. In one preferred embodiment, L-lysine-d-amphetamine is administered to patients in an amount of from about 20 mg to about 70 mg/day (e.g., 20 mg, 30 mg, 40 mg, 50 mg, 60 mg, 70 mg/day) based on the amount of L-lysine-d-amphetamine dimesylate. In another embodiment, L-lysine-d-amphetamine is administered in an amount of from about 15 mg to about 35 mg/day (e.g., about 16 mg/day or 32 mg/day). In an alternative embodiment, L-lysine-d-amphetamine is administered to patients in a range equivalent to a dose range of from about 10% to about 90%, from about 15% to about 80%, from about 20% to about 50% of the currently effective doses (e.g., 70 mg/day). In this aspect, L-lysine-d-amphetamine is administered in a range equivalent to a dose range of from about 15 mg to about 35 mg/day of L-lysine-d-amphetamine dimesylate (e.g., about 16 mg/day or 32 mg/day).

In a preferred aspect of the invention, the methods described herein provide a method for treating depressive disorder with an SSRI such as escitalipram or sertraline in amounts of less than the currently effective daily doses in combination with L-lysine-d-amphetamine. In one embodiment, escitalopram (e.g., escitalopram oxalate) administered to patients is in a range of from about 10% to about 50% (e.g., from about 10% to about 45%, from about 20% about 45%, from about 30% to about 45%) of the currently effective daily dose of about 20 mg/day of escitalopram oxalate. In another embodiment, sertraline can be administered to patients in a range of from about 5% to about 10%, from about 8% to about 10%, from about 25% to about 32%, from about 25% about 32% of the currently effective daily dose of about 50 mg/day of sertraline.

In a related embodiment, the invention includes a method of increasing the antidepressant effect of a selective serotonin reuptake inhibitor in mammals. The method includes administering the same combination of SSRI and amphetamine prodrugs in the amounts recited above. Preferred aspects of this embodiment include administering an effective amount of escitalopram, or a pharmaceutical salt thereof, in combination with an effective amount of L-lysine-d-amphetamine, or a pharmaceutically acceptable salt thereof.

A still further embodiment of the invention includes methods of enhancing or potentiating the therapeutic effects of SSRI's in mammals. The methods include administering an effective amount of an SSRI to a mammal having an SSRI-treatable condition in combination with an amount of an amphetamine prodrug which is sufficient to enhance or potentiate the SSRI effects in the mammal.

### Dosage Forms

Both the SSRI and amphetamine prodrug will be administered using commonly available dosage forms. In many aspects, the SSRI and amphetamine prodrug will be administered in separate dosage forms to the mammal in need thereof. In other aspects, the two agents will be provided in a single dosage form which includes the combination. A non-limiting list of suitable dosage forms includes, for example, tablets, coated tablets, dragees, capsules, hard gelatine capsules, soft gelatine capsules, caplets, lozenges, oral solutions, oral suspensions or combinations thereof. The active ingredients may be mixed under sterile conditions with a pharmaceutically acceptable carrier and may be in aqueous or non-aqueous forms.

Preferred dosage forms are oral dosage forms such as tablets, capsules, caplets and lozenges. These improve patient compliance relative to other dosage forms.

The dosage forms may also contain any carriers or excipients such as diluents, binders and adhesives, lubricants, plasticizers, disintegrants, colorants, bulking substances, flavoring, sweeteners, buffers, adsorbents, etc. required for making a pharmaceutically acceptable dosage. For instance, the carriers or excipients may include microcrystalline cellulose, lactose, corn starch or derivatives thereof, talc, stearic acid or its salts etc. Suitable carrier materials for soft gelatine capsules can include, for example, vegetable oils, waxes, fats and semi-solid and liquid polyols. Suitable carrier materials for the production of solutions and syrups include, for example, water, polyols, sucrose, invert sugar and the like.

L-lysine-d-amphetamine was evaluated alone and in combination with a number of SSRIs at varying dosages to evaluate its potential antidepressant activity and to evaluate augmentation effects. In this study, augmentation is defined as the observation of a better effect with the combination of L-lysine-d-amphetamine together than is observed for either individual component at the same dose. The two active agents in the combination act in a different manner and hence the dosages are not additive. Consequently, an improvement relative to the individual dosages demonstrates synergy or augmentation

Male Rj: NMRI mice (Elevage Janvier, 53940 Le Genest-Saint-Isle, France) having a body weight of between 22 and 29 g were used in the study. The mice were stabilised for 5 days prior to assessment with free access to food and water. The test substances were administered 90 minutes (behaviour despair test) before the test. The test results shown in Table 2 express the dosages in mg/kg of supplied substance. The control vehicle was distilled water. The substances were evaluated in comparison with the control vehicle and were administered dissolved in distilled water.

### Experimental Procedure

The method, which detects antidepressant activity, follows that described by Porsolt et al (Arch. Int. Pharmacodyn., 1977, 229, 327-336). Mice forced to swim in a situation from which they cannot escape rapidly become immobile. Antidepressants decrease the duration of immobility.

Mice were individually placed in a cylinder (height=24 cm; diameter=13 cm) containing 10 cm water (22° C.) from which they cannot escape. The mice were placed in the water for 6 minutes and the duration of immobility during the last 4 minutes was measured. The latency to the first bout of immobility was also recorded.

10 mice were studied per group. The test was performed blind.

L-lysine-d-amphetamine (Vyvanse) (8, 16 and 32 mg/kg) was administered 90 minutes before the test, alone or in combination and compared with a vehicle control group. Doses were selected as those that were sub-threshold based upon results in a previous study.

Imipramine (128 mg/kg p.o.), administered under the same experimental conditions, was used as a reference substance.

Data were analysed by comparing treated groups with vehicle control using unpaired Student's t tests. In addition, groups treated with combinations of substances were compared with groups administered appropriate substances alone using unpaired Student's t tests.

### EXAMPLES

The examples which follow illustrate exemplary embodiments of the methods of the present invention.

### Example 1

### Effect of L-Lysine-D-Amphetamine in Combination with Escitalopram on Mouse Behavioural Despair Test

The mouse behavioural despair test is based on the observation that mice, when forced to swim in a situation from which there is no escape, after an initial period of vigorous activity will eventually cease to move altogether making only those movements necessary to keep the head above the water. This behavioural immobility is thought to indicate a state of despair in which the mice have learned that escape is impossible and have resigned themselves to the experimental conditions. This immobility is reduced by anti-depression treatments.

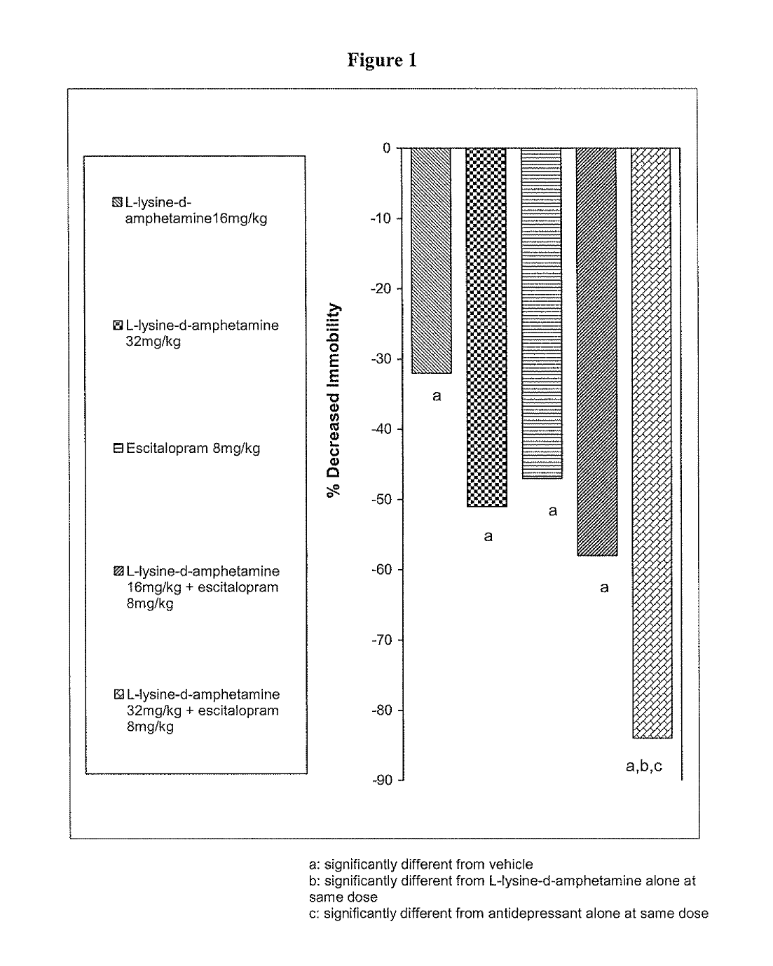

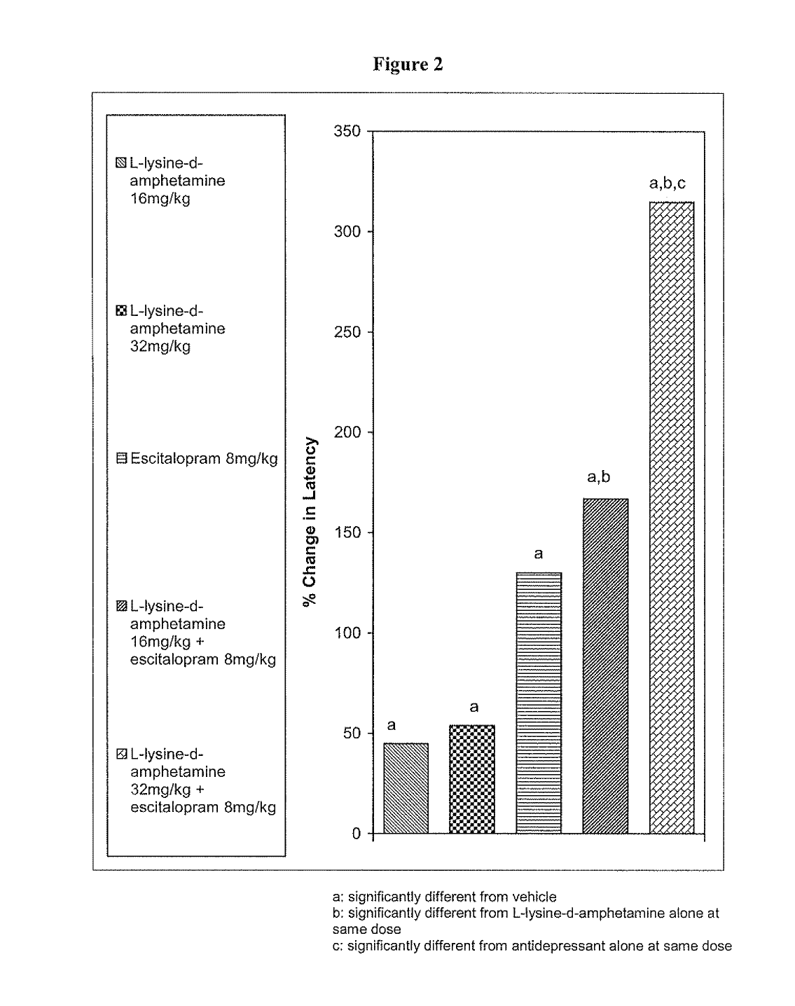

Drugs and doses investigated are shown in Table 1 while the results of the experiment are shown in FIGS. 1 and 2.

TABLE 1

| Drug | Oral Dose |
|---|---|
| L-lysine-d-amphetamine | 16 mg/kg |
| L-lysine-d-amphetamine | 32 mg/kg |
| Escitalopram | 8 mg/kg |
| L-lysine-d-amphetamine + escitalopram | 16 mg/kg + 8 mg/kg |
| L-lysine-d-amphetamine + escitalopram | 32 mg/kg + 8 mg/kg |

FIG. 1 shows the duration of immobility among the different experimental groups. Mice treated with the combination of L-lysine-d-amphetamine and escitalopram exhibited a reduced immobility time. Specifically, oral administration 60 minutes before the test of a combination of L-lysine-d-amphetamine (16 and 32 mg/kg) with escitalopram (8 mg/kg), markedly and dose-dependently decreased the duration of immobility, as compared with vehicle control (−58% and −84%, respectively, p<0.001). The effects of L-lysine-d-amphetamine at 16 and 32 mg/kg combined with escitalopram at 8 mg/kg were more marked than the effects of each substance administered alone.

The results show that decreased immobility time is specifically achieved only when L-lysine-d-amphetamine is administered in combination with a selective serotonin reuptake inhibitor.

In conclusion, the results from the immobility test demonstrated that immobility was remarkably reduced in mice treated orally with L-lysine-d-amphetamine in combination with escitalopram.

FIG. 2 shows the time to latency of immobility among the different experimental groups. Specifically, L-lysine-d-amphetamine+escitalopram markedly and dose-dependently increased the latency to immobility (+167%, p<0.01 and +316%, p<0.001, respectively).

The time to latency is also specifically achieved by L-lysine-d-amphetamine administered in combination with a selective serotonin reuptake inhibitor.

In conclusion, the results of measurement of the latency to immobility confirmed that latency of immobility was significantly increased in mice treated with L-lysine-d-amphetamine combined with escitalopram.

### Example 2

### Effects of L-Lysine-D-Amphetamine in Combination with Escitalopram on the Light-Dark Box Test of Anxiety

The light/dark test is based on the innate aversion of rodents to brightly/illuminated areas and on the spontaneous exploratory behaviour of rodents in response to mild stress, that is, novel environment and light. The test apparatus consists of a compartment box divided between a dark safe compartment and an illuminated aversive compartment.

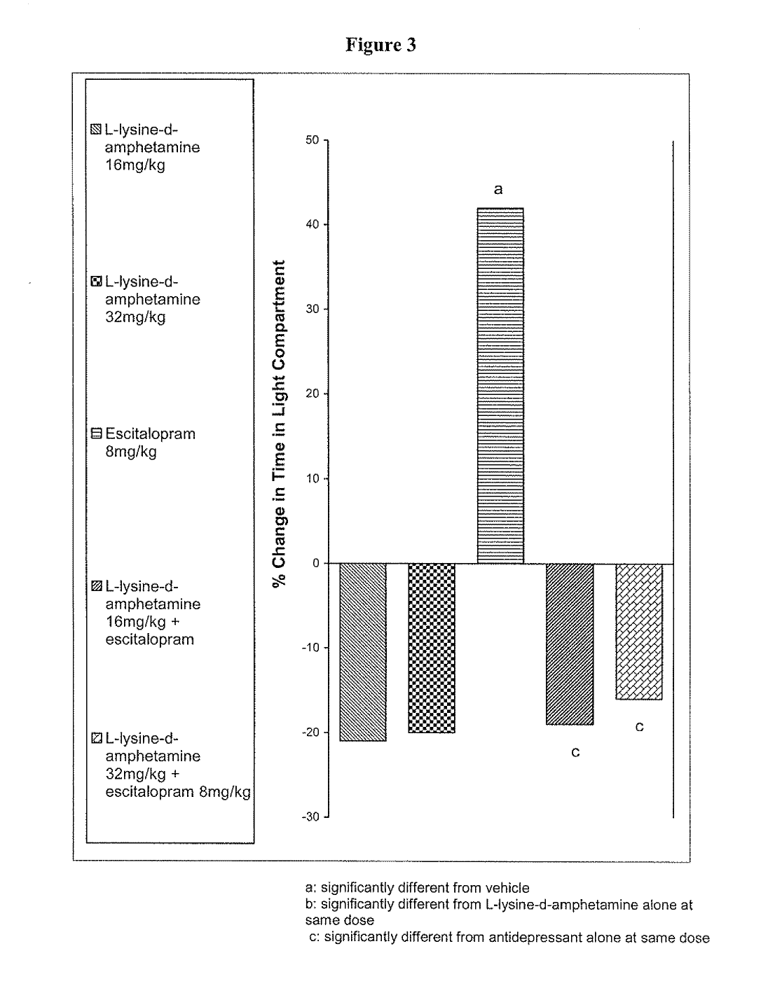

The drugs and the doses investigated are the same shown in Table 1 above. FIG. 3 shows that L-lysine-d-amphetamine (16 and 32 mg/kg p.o.) administered in combination with escitalopram (8 mg/kg) before the test, did not affect the time spent in the light compartment, as compared with vehicle control nor did it affect the number of crossings. However, the effects (time spent) of L-lysine-d-amphetamine at 16 and 32 mg/kg combined with escitalopram at 8 mg/kg were significantly different from the effects of escitalopram alone.

These results suggest the presence of antidepressant-like activity and the absence of any anxiogenic activity in mice treated with L-lysine-d-amphetamine at 16 and 32 mg/kg, in both the Behavioural Despair and Light-Dark Box Tests. Escitalopram at 8 mg/kg had moderate antidepressant and weak anxiolytic activity in the same tests. The combination of L-lysine-d-amphetamine (16 and 32 mg/kg) and escitalopram (8 mg/kg) increased antidepressant activity, as compared with the effects of the two substances administered alone.

Table 2 below provides further data in the behaviour despair study in mice. This data shows significant augmentation effects for combinations of L-lysine-d-amphetamine with escitalopram.

TABLE 2
Effects of vyvanse, escitalopram, and imipramine in the
Behavioral Despair Test in the mouse (10 mice per group)

| TREATMENT | DURATION OF IMMOBILITY | | | | | | LATENCY | | | | | |
|---|---|---|---|---|---|---|---|---|---|---|---|---|
| (mg/kg) | (s) | | | | | | (s) | | | | | |
| p.o. -60 min | mean ± s.e.m. | | | p value | % change | | mean ± s.e.m. | | | p value | % change | |
| Vehicle | 213.2 ± 6.0 | | | | | | 64.7 ± 5.6 | | | | | |
| Vyvanse (16) | 173.6 ± 18.8 | NS | (a) | 0.0598 | −19% | (a) | 100.0 ± 21.3 | NS | (a) | 0.1268 | +55% | (a) |
| Vyvanse (32) | 160.3 ± 22.6 | * | (a) | 0.0363 | −25% | (a) | 104.3 ± 18.2 | NS | (a) | 0.0517 | +61% | (a) |
| Escitalopram (2) | 184.8 ± 8.3 | * | (a) | 0.0123 | −13% | (a) | 108.2 ± 13.2 | ** | (a) | 0.0071 | +67% | (a) |
| Escitalopram (4) | 182.5 ± 9.3 | * | (a) | 0.0123 | −14% | (a) | 105.0 ± 8.8 | ** | (a) | 0.0011 | +62% | (a) |
| Vyvanse (16) + | 159.8 ± 21.1 | * | (a) | 0.0256 | −25% | (a) | 95.4 ± 8.1 | ** | (a) | 0.0057 | +47% | (a) |
| Escitalopram (2) | | NS | (b) | 0.6312 | −8% | (b) | | NS | (b) | 0.8424 | −5% | (b) |
| | | NS | (c) | 0.2848 | −14% | (c) | | NS | (c) | 0.4187 | −12% | (c) |
| Vyvanse (16) + | 126.6 ± 19.8 | *** | (a) | 0.0006 | −41% | (a) | 156.6 ± 18.9 | *** | (a) | 0.0002 | +142% | (a) |
| Escitalopram (4) | | NS | (b) | 0.1025 | −27% | (b) | | NS | (b) | 0.0623 | +57% | (b) |
| | | * | (c) | 0.0200 | −31% | (c) | | * | (c) | 0.0233 | +49% | (c) |
| Vyvanse (32) + | 182.2 ± 18.2 | NS | (a) | 0.1222 | −15% | (a) | 118.8 ± 18.3 | * | (a) | 0.0112 | +84% | (a) |
| Escitalopram (2) | | NS | (b) | 0.4599 | +14% | (b) | | NS | (b) | 0.5812 | +14% | (b) |
| | | NS | (c) | 0.8978 | −1% | (c) | | NS | (c) | 0.6445 | +10% | (c) |
| Vyvanse (32) + | 85.3 ± 22.3 | *** | (a) | <0.0001 | −60% | (a) | 159.3 ± 31.7 | ** | (a) | 0.0088 | +146% | (a) |
| Escitalopram (4) | | * | (b) | 0.0297 | −47% | (b) | | NS | (b) | 0.1499 | +53% | (b) |
| | | *** | (c) | 0.0008 | −53% | (c) | | NS | (c) | 0.1165 | +52% | (c) |
| Imipramine (128) | 70.4 ± 21.4 | *** | (a) | <0.0001 | −67% | (a) | 243.6 ± 32.7 | | (a) | <0.0001 | +277 | (a) |

Student's t test: NS = Not Significant;
* = p < 0.05;
** = p < 0.01;
*** = p < 0.001.
(a): compared with vehicle control.
(b): compared with vyvanse alone at the same dose.
(c): compared with escitalopram alone at the same dose.

Table 3 below provides further data in the behaviour despair study in mice. This data shows significant augmentation effects for combinations of L-lysine-d-amphetamine with sertraline.

TABLE 3
Effects of vyvanse, sertraline and imipramine in the Behavioral
Despair Test in the mouse (10 mice per group)

| TREATMENT | DURATION OF IMMOBILITY | | | | | |
|---|---|---|---|---|---|---|
| (mg/kg) | (s) | | | | | |
| p.o. -90 min | mean ± s.e.m. | | | p value | % change | |
| Vehicle | 193.9 ± 15.9 | | | | | |
| Vyvanse (8) | 208.0 ± 7.3 | NS | (a) | 0.4310 | +7% | (a) |
| Vyvanse (16) | 176.8 ± 14.8 | NS | (a) | 0.4419 | −9% | (a) |
| Vyvanse (32) | 123.1 ± 20.8 | * | (a) | 0.0146 | −37% | (a) |
| Sertraline (16) | 162.7 ± 27.5 | NS | (a) | 0.3388 | −16% | (a) |
| Vyvanse (8) + | 125.6 ± 25.2 | * | (a) | 0.0342 | −35% | (a) |
| Sertraline (16) | | ** | (b) | 0.0057 | −40% | (b) |
| | | NS | (c) | 0.3331 | −23% | (c) |
| Vyvanse (16) + | 91.1 ± 15.1 | *** | (a) | 0.0002 | −53% | (a) |
| Sertraline (16) | | *** | (b) | 0.0008 | −48% | (b) |
| | | * | (c) | 0.0348 | −44% | (c) |
| Vyvanse (32) + | 117.9 ± 27.8 | * | (a) | 0.0290 | −39% | (a) |
| Sertraline (16) | | NS | (b) | 0.8826 | −4% | (b) |
| | | NS | (c) | 0.2667 | −28% | (c) |
| Imipramine (128) | 100.6 ± 23.5 | ** | (a) | 0.0041 | −48% | (a) |

Student's t test: NS = Not Significant;
* = p < 0.05;
** = p < 0.01;
*** = p < 0.001.
(a): compared with vehicle control.
(b): compared with vyvanse alone at the same dose.
(c): compared with sertraline alone at the same dose.

In conclusion, these results suggest that an amphetamine prodrug such as L-lysine-d-amphetamine at doses causing relatively minimal overt CNS effects augments the antidepressant effect of the selective serotonin reuptake inhibitor escitalopram and sertraline.

1. A method of treating depression in a mammal, comprising administering an effective amount of a selective serotonin reuptake inhibitor or a pharmaceutically acceptable salt thereof in combination with an amphetamine prodrug, or pharmaceutically acceptable salt thereof to a mammal in need thereof, wherein the selective serotonin reuptake inhibitor is citalopram, escitalopram, paroxetine and sertraline, or a pharmaceutically acceptable salt thereof.

2. The method of claim 1, wherein the amphetamine prodrug is an amino acid prodrug of amphetamine or a pharmaceutically acceptable salt thereof.

3. The method of claim 2, wherein the amino acid prodrug is L-lysine-d-amphetamine or a pharmaceutically acceptable salt thereof.

4. The method of claim 1, wherein the serotonin reuptake inhibitor is selected from escitalopram and sertraline or pharmaceutically acceptable salts thereof.

5. The method of claim 4, wherein the serotonin reuptake inhibitor is escitalopram or a pharmaceutically acceptable salt thereof.

6. The method of claim 3, wherein the effective amount of L-lysine-d-amphetamine is from about 20 mg to about 70 mg/day.

8. The method of claim 1, wherein the effective amount of escitalopram comprises from about 10 to about 20 mg/day.

9. The method of claim 1, wherein the selective serotonin reuptake inhibitor and the amphetamine prodrug composition are administered in a single pharmaceutical composition.

10. The method of claim 9, wherein the pharmaceutical composition is in a form selected from the group consisting of tablets, coated tablets, dragees, capsules, hard gelatine capsules, soft gelatine capsules, caplets, oral solutions, oral suspensions or combination thereof.

11. The method of claim 1, wherein the serotonin reuptake inhibitor is escitalopram or a pharmaceutically acceptable salt thereof and the amphetamine prodrug is L-lysine-d-amphetamine or a pharmaceutically acceptable salt thereof.

12. The method of claim 11, wherein the escitalopram or pharmaceutically acceptable salt thereof is administered to a patient in a range of from about 10% to about 50% of the currently effective daily dose of escitalopram.

13. The method of claim 12, wherein the amphetamine prodrug is administered to a patient in a range equivalent to a dose range of from about 20% to about 50% of the currently effective daily dose of L-lysine-d-amphetamine dimesylate.

14. A method of increasing the antidepressant effect of a selective serotonin reuptake inhibitor in a mammal, comprising administering an effective amount of a selective serotonin reuptake inhibitor or a pharmaceutically acceptable salt thereof, in combination with an effective amount of an amphetamine prodrug to a mammal in need thereof.

15. A method enhancing the therapeutic effects of a selective serotonin reuptake inhibitor in a mammal, comprising administering an effective amount of a selective serotonin reuptake inhibitor or a pharmaceutically acceptable salt thereof to a mammal having an SSRI-treatable condition in combination with an amount of an amphetamine prodrug which is sufficient to enhance or potentiate the SSRI effects in the mammal.

16. A kit for use in the treatment of a patient having depression, comprising a selective serotonin reuptake inhibitor selected from the group comprising: citalopram, escitalopram, paroxetine and sertraline or a pharmaceutically acceptable salt thereof formulated for administration to the patient and an amphetamine prodrug or a pharmaceutically acceptable salt thereof formulated for administrated to the patient.
